(19) United States
(12) Patent Application Publication
(10) Pub. No.: US 2025/0285743 A1
(43) Pub. Date: Sep. 11, 2025

(54) Zero-Touch Scheduling Application

(71) Applicant: Thomas R. Campi, JR., Wilmington, DE (US)

(72) Inventor: Thomas R. Campi, JR., Wilmington, DE (US)

(21) Appl. No.: 19/072,576

(22) Filed: Mar. 6, 2025

Related U.S. Application Data
(60) Provisional application No. 63/561,899, filed on Mar. 6, 2024.

(51) Int. Cl. G16H 40/20

(52) U.S. Cl. CPC G16H 40/20

(57) ABSTRACT

A scheduling application is implemented that uses an internal medical provider's system or remote service to gather patient data, including clinical severity and health information, for smarter appointment management. The system prioritizes high-need patients and fills newly opened slots due to cancellations or late arrivals by assessing severity levels from clinician input, clinical factors, or AI evaluations. Providers can choose between an “auto-fill” function that instantly schedules these openings or a “manual fill” approach for greater control. Through intuitive interfaces showing provider availability, patient severity, and insurance details, the application notifies both patients and providers in real-time, minimizing no-shows and ensuring prompt, efficient care delivery.

### CROSS-REFERENCES TO RELATED APPLICATIONS

This Non-Provisional Utility Patent Application claims the benefit of and priority to U.S. Provisional Application Ser. No. 63/561,899, filed Mar. 6, 2024, entitled “Scheduling Assistant,” the entire contents of which is hereby incorporated herein by reference.

### BACKGROUND

It is widely accepted that up to a quarter, or even half, of all deaths are preventable. However, patients who miss medical and mental health appointments, and do not follow up disease prevention create high and avoidable costs. When diseases are not detected early due to missed screenings or follow-ups, they can progress to more advanced stages requiring more expensive treatments like surgeries or intensive therapies. Telehealth and online booking have helped reduce barriers to care by decreasing patient no-shows and expanding appointment flexibility. However, current systems still lack the ability to fill newly opened slots immediately, causing not only wasted clinical provider time-but delays access for those waiting for care (especially in specialist fields) and lacks a systemized way of following up and scheduling for missed appointments so patient's do not fall out of care and letting their health deteriorate. Wait times excruciating long-making untreated disease harder to treat and patient's often give up-as it's a cited that 1% give up for each day of wait. A system that also provides access to providers is also needed and fulfills President Trump's “Make America Healthy Again” initiative and mandate to cut down on waste and abuse within Medicaid.

A dynamic scheduling tool that factors in patient severity, wait times, provider availability, and appointment histories can overcome these limitations. By providing providers with clinical prioritization and instantly notifying patients of openings via digital alerts, the system can ensure prompt treatment and limit revenue losses from missed appointments. Automating and prioritizing scheduling decisions also aligns with legal and ethical duties to care for high-severity patients while meeting regulatory requirements for timely access to care.

### SUMMARY

This scheduling application leverages an internal system or remote service that collects and processes patient data, including clinical severity and related health information, to optimize appointment scheduling. The system prioritizes patients based on severity levels, which can be determined through clinician input, historical records, or AI driven assessments. By integrating with provider computing devices, the scheduling application dynamically proposes schedule modifications—filling newly available slots caused by cancellations or late arrivals and ensuring high-severity patients receive prompt care.

To facilitate streamlined workflows, the scheduling application supports both automated and manual booking. An “auto-fill” function can immediately populate open time slots when the system detects available capacity, while a “manual fill” option lets providers confirm or adjust bookings. Graphical user interfaces for example shows clinical relevant criteria such as show a provider's openings, as well as patient details such as severity ratings, insurance requirements, and previous appointment history. Through real-time notifications to patients and providers, this system reduces openings, no-shows, cancellations, improves healthcare access, and aligns patient need with provider availability in a responsive, efficient manner.

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure. It will be appreciated that the above-described subject matter may be implemented as a computer-controlled apparatus, a computer process, a computing system, or as an article of manufacture such as one or more computer-readable storage media. These and various other features will be apparent from reading the following Detailed Description and reviewing the associated drawings.

### BRIEF DESCRIPTION OF THE DRAWINGS

FIG. 1 shows an illustrative flowchart of a scheduling assistant system as disclosed herein;

FIG. 2 shows an illustrative environment of a remote scheduling assistant service or internal medical provider's service and their respective scheduling applications receiving patient clinical data and modifying doctor-patient schedules;

FIG. 3 shows an illustrative representation of the remote service assessing a schedule modification based on various factors;

FIG. 4 shows an illustrative graphical user interface (GUI) representation of the scheduling application with openings and auto-fill functionality enabled; [11] FIG. 5 shows an illustrative GUI representation of the scheduling application with the openings filled with patient visits and a manual fill auto-fill option for a user;

FIG. 6 shows an illustrative representation of the manual fill implementation;

FIG. 7 shows an illustrative GUI representation of the auto-fill implementation in an alternate approach;

FIG. 8 shows an illustrative GUI representation of a provider's simultaneous use of the auto-fill option and manual fill option, depending on the opening; FIG. 9 shows an illustrative GUI representation of the scheduling application pulling data from an internal medical and/or scheduling system;

FIG. 10 shows an illustrative GUI representation of a medical provider's dashboard for the scheduling application;

FIG. 11 shows an illustrative GUI representation of the provider's scheduling

application on the dashboard being updated while on the dashboard. [18] FIG. 12 is a simplified block diagram of an illustrative architecture of a computing device that may be used at least in part to implement the present scheduling application;

FIG. 13 is a simplified block diagram of an illustrative remote computing device, remote service, internal remote service, or computer system that may be used in part to implement the present scheduling application;

FIGS. 14-17 show illustrative representations of a computing device, such as a smartwatch, providing various additional features such as assessing patient clinical factors and enabling scheduling of patient visits; and

FIG. 18 shows an illustrative representation of an overall systemized health network.

Like reference numerals indicate like elements in the drawings. Elements are not drawn to scale unless otherwise indicated.

### DETAILED DESCRIPTION

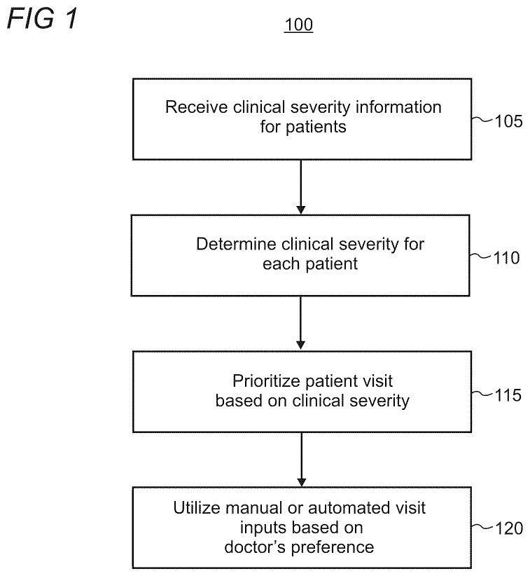

FIG. 1 shows an illustrative flowchart 100 providing a high-level overview of the scheduling application discussed herein. In step 105, an internal system or remote service may receive clinical severity information for patients. Clinical severity information may be any information about patients, whether based on patient inputs/complaints, provider information, etc. In step 110, the internal system or the remote service may determine a clinical severity for each patient based on the received clinical severity information. Such clinical severity information can include a rating of the patient's severity on a numeral scale (e.g., 110), a categorical scale such as low, medium, and high severity, or another rating system. Such information can be based on clinical factors about the patient, a provider's determined rating, or even AI (Artificial Intelligence)-based using known patient information (e.g., symptoms, medical/mental health complaints, patient history, treatment adherence, socio-economic factors, etc.).

In step 115, internal system or the remote service may prioritize counseling sessions based on the determined clinical severity per patient. Patients with greater indicated severity symptoms may be ranked higher for treatment services on a provider's schedule to be seen sooner, whereas patients with less severity may be ranked lower and receive a provider session relatively later. In step 120, the internal system or remote service interoperating with local scheduling applications on provider computing devices may utilize manual or automated counseling sessions into a provider's calendar of patient's visits. Such automated or manual functions may be based on the specific provider's or provider's staff's preferences input into the scheduling application.

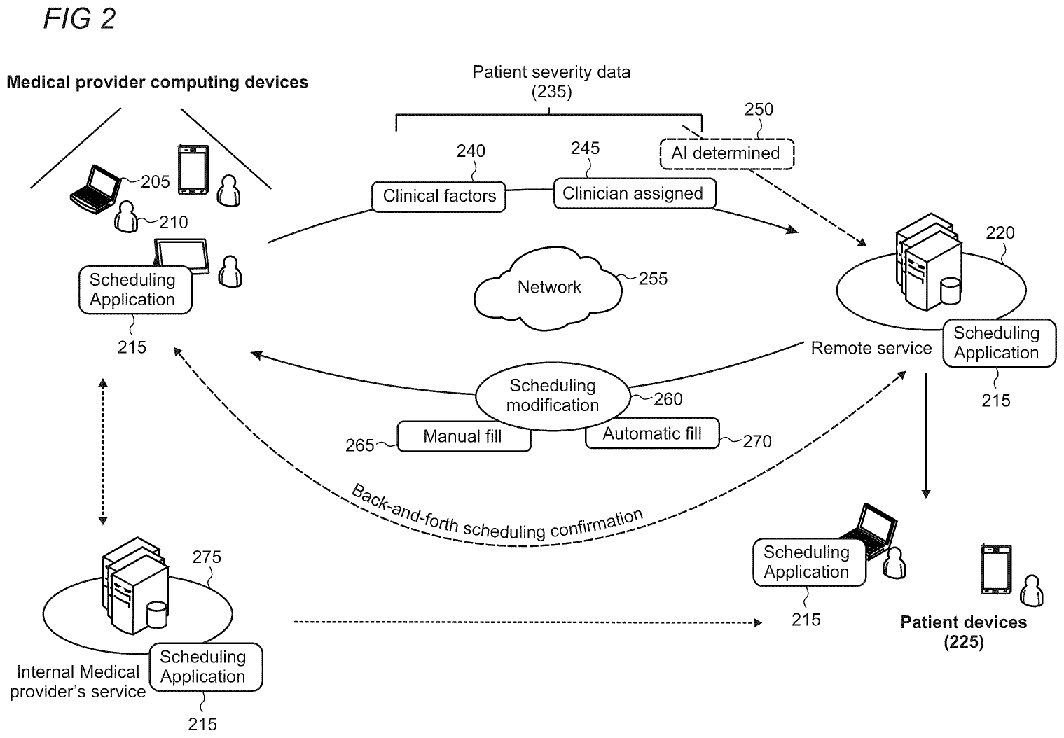

FIG. 2 shows an illustrative environment in which the internal medical provider's service 275 or remote service 220 may communicate with medical provider computing devices 205 operated by providers and patient devices 225 operated by patients. Each may have a scheduling application 215 instantiated on their computing devices. While a dedicated scheduling application is discussed herein, it should be understood that the scheduling application may be accessed via a web browser to uniform resource locator that performs similar functions as the scheduling application.

While the discussion herein focuses on the remote service 220 operating the scheduling application 215 and communications with patient devices 225 and medical provider computing devices 205, it should be understood, as exemplified in FIG. 2, that the scheduling application may alternatively or additionally be fully internalized on the medical provider's internal system or service 275. Thus, for example, a hospital may update their internal systems with the scheduling application and all of the features discussed herein, without the use of the remote service 220. In this regard, any discussion of the remote service 220 performing operations and communicating with the medical provider's computing devices 205 or patient devices 225, or processes, functions, GUIs (graphical user interfaces) shown throughout the drawings, may alternatively be performed solely in-house by a given medical provider. Alternatively, the internal medical provider's service 275 may interoperate with the remote service 220, such that, for example, the remote service updates the medical provider's scheduling application 215 with periodic updates to enhance their system.

The medical including mental health provider computing devices 205 may be devices operated by individual medical professionals or providers, or alternatively can be part of an internal system within a hospital, provider's office, or other medical setting, Thus, the scheduling application 215 and remote service 220 (or internal service 275) may interoperate with EMRS (Electronic Medical Record System), EMHS (Electronic Mental Health Systems), or other scheduling or proprietary medical system utilized by the medical provider and business.

The medical provider computing device(s) 205 may transmit various patient and provider data to the remote service 220 or internal medical provider's service 275. For example, patient severity data 235 can be transmitted to the internal system or remote service so that the internal system or remote service can utilize such information in scheduling decisions and modifications. Patient severity data 235 may include, for example, clinical factors 240, clinician assigned 245, or AI determined 250. Clinical factors may include, for example, medical history, symptoms and medical/mental health complaints, screens, scores, vital signs, lab results, medications and allergies, mental health indicators, lifestyle factors, etc. The AI determined option may occur using the clinical factors with or without the clinician assigned patient severity.

The internal system's or remote service's scheduling application 215 may digest such inputs and transmit a schedule modification 260 to the medical/mental health provider computing devices 205. Such schedule modifications may, based on the specific provider's preference, be manually filled 265 into their schedule or auto-filled 270. Such modifications may result in some back-and-forth communications between the provider and internal system or remote service to verify certain patient bookings, switch patients, etc. If the patient operates a scheduling application 215 on their patient devices 225, then they may get a notification as well about their scheduled appointment (e.g., day and time, provider's name, location, etc.). In this regard, the scheduling application may be a standalone application instantiated on the patient's computing device, a URL accessed via a browser, or be a plugin to their medical provider's own internal application or system, such as via the provider's website, application, etc.

The various communications may occur over the network 255. The network can include any one or more of a local area network (LAN), wide area network (WAN), the Internet, or the world wide web.

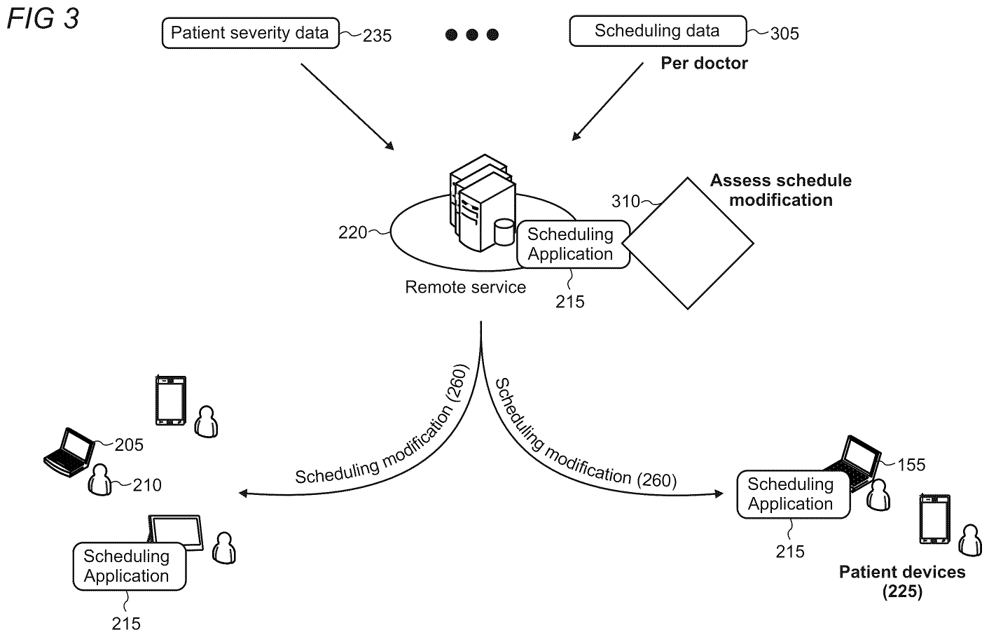

FIG. 3 shows an illustrative environment in which the remote service's scheduling application 215, or internal medical provider's service 275, receives various data in assessing schedule modifications. For example, the internal system or remote service may receive the patient severity data 235 (FIG. 1), scheduling data 305 for providers, and other data that may be relevant for making determinations. For example, the internal system or remote service may interoperate with an AI engine so that upon receiving clinical factor data 240, it can make a refined determination for scheduling and patient severity answers. The scheduling data may be the provider's schedule, such as filled slots, empty slots, patient information within the calendar, among other data.

Upon receiving this data, the remote service 220 may utilize the scheduling application 215 to assess schedule modifications 310 for each provider. Such a determination may be made because, for example, patients may cancel meetings with little to no notice, or provider schedules may just have empty slots. The scheduling application attempts to make such proposed schedule modifications, in real-time, so that the providers schedule is fully utilized and patients are receiving the care they need as soon as possible-whether severity level is utilized or not. Rating patients by severity level ensures that they are getting the help they need as early as possible, especially when a provider's schedule opens up.

Upon the internal system or remote service assessing a determining a schedule modification at step 310, the internal system or remote service 220 transmits the proposed scheduling modification 260 to the medical/mental health provider computing devices 205 and, if feasible, the patient computing devices 225. Alternatively, the internal system or remote service may transmit the scheduling modification to the patient once the provider confirms or approves the appointment.

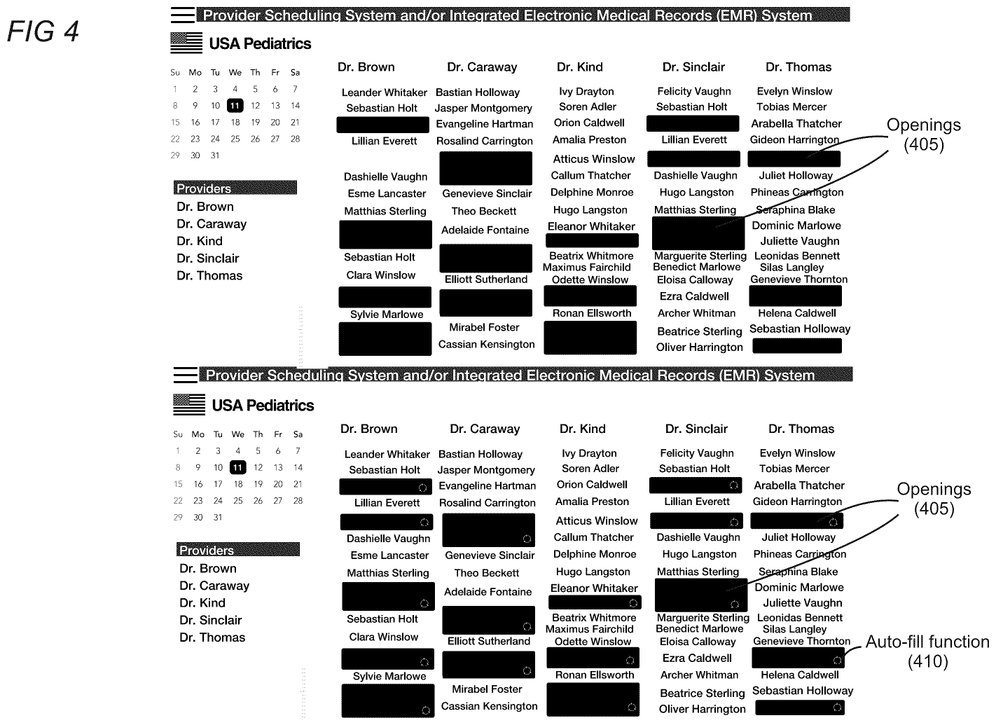

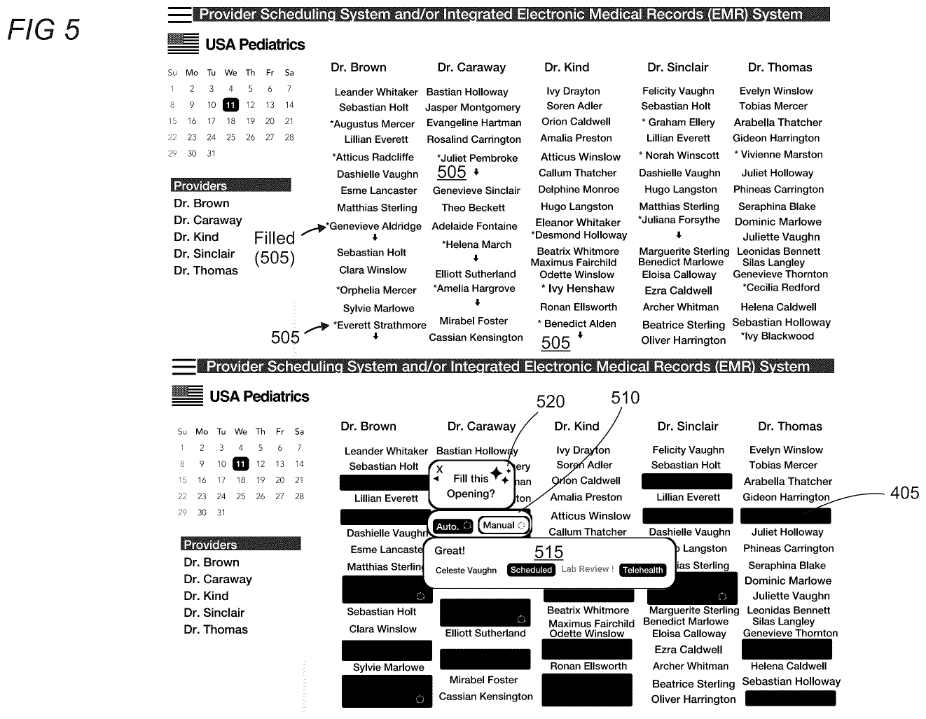

FIG. 4 shows an illustrative GUI (graphical user interface) of a provider's internal scheduling system or one that is integrated with an EMRS. Such a GUI may be implemented within a scheduling application 215 that is integrated with a provider's proprietary system or the EMRS. In this regard, the scheduling application may be a plugin to the provider's own internal system.

The GUI shows the daily schedule for a series of medical providers. Specifically, the GUI shows various openings 405 in black. The openings are dates and times that the provider has no scheduled patient appointments. The lower end of the GUI shows each opening 405 configured with the auto-fill function 410. The auto-fill function enables the proposed scheduling modifications 260 from the remote service 220 or internal service 275 to be automatically propagated into the provider's open slots. Thus, any of the auto-fill-enabled openings would be automatically filled with a patient responsive to the internal system or remote service's proposal.

FIG. 5 shows an illustrative GUI representation in which the various openings 405 for the individual providers have been filled 505 responsive to receiving the scheduling modification 260 from the internal service 275 or remote service 220. The bottom portion of FIG. 5 shows a proposed GUI with pop-up boxes where the user can select the “auto” or “manual” in box 510, the system can verify to “fill this opening” 505, and a confirmation 515 box shows the scheduled appointment and the patient's name in a given opening slot. It should be noted that fake patient names have been illustrated.

Patients pay be propagated into a provider's scheduling application 215 when the scheduling application knows that the patient needs to be seen, or based on the patient's provided available times. TOM I CLEANED UP->For example, the internal system or remote service automatically schedules a patient to be seen—requesting soon care or re-scheduled for a missed follow-up or preventative health visit, or a medical/mental health provider puts in a patient in when available—these may be existing patients or a new patient that has registered with the medical provider and is awaiting a visit. For example, some patients, such as psychiatric/mental health patients, may regularly see a provider. Those patients may be stored within the system and placed into a provider's schedule according to the present. Alternatively, the patients may provide their availability to the medical provider, which may be used by the scheduling application 215 such as at the internal service 275 or remote service 220. When the patient's schedule overlaps with a provider's the scheduling application may input that patient into the opening 405. This may be done with the auto-fill or manual fill options. So, as discussed below, the auto-fill option may automatically select that patient if they are actually available for their time, and the manual fill option may present that patient as at least one available patient for that time.

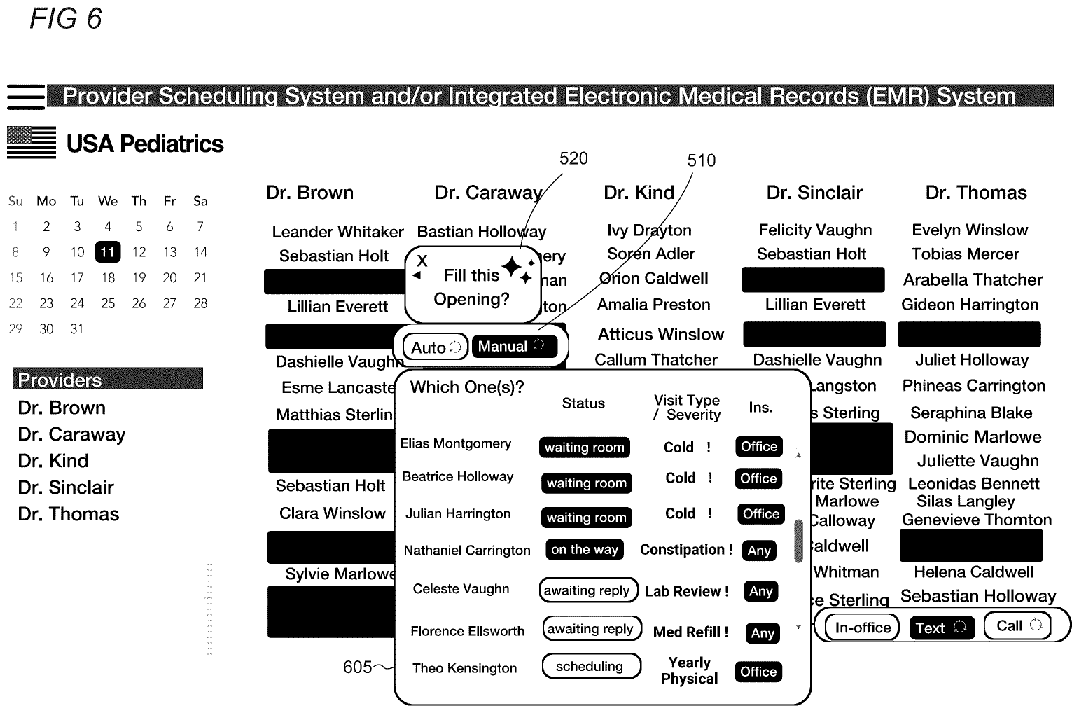

FIG. 6 shows an illustrative GUI representation in which the user-provider can change the “auto” or “manual” box 510 to “manual.” This will enable the provider to click on an opening 405, be presented with some available patient data in box 605 to select a patient to fill that opening. Upon selecting a patient from the list, the scheduling application with or without the internal system's or remote service's aid may communicate with a patient device 225 to relay the scheduled appointment and/or to receive confirmation that the patient wishes to attend the session at that time.

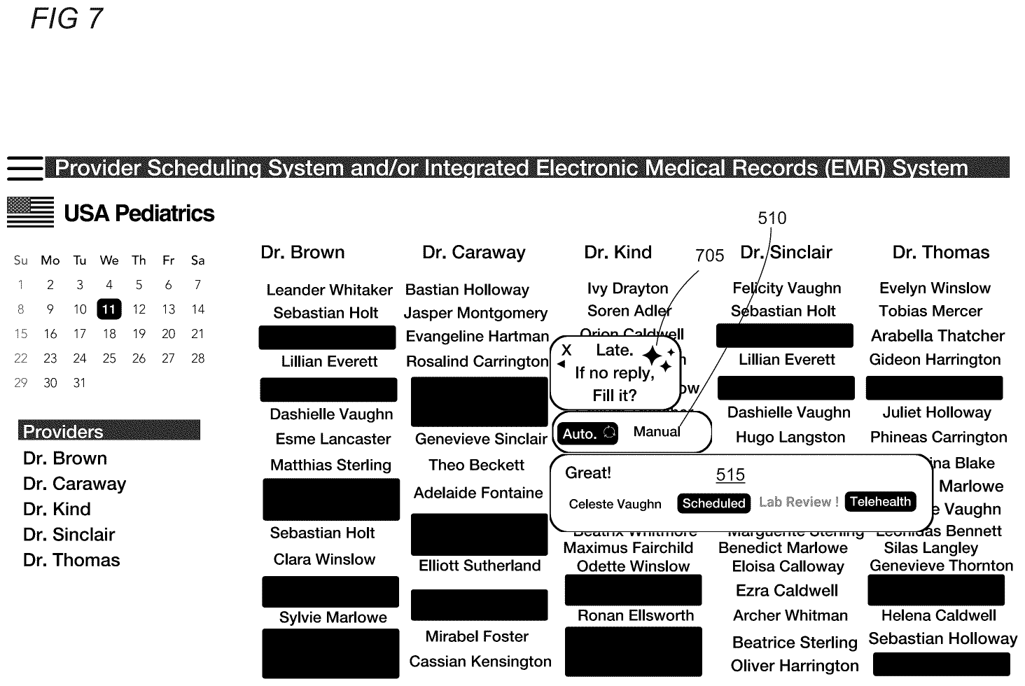

FIG. 7 shows an illustrative GUI representation in which the scheduling application 215 implements processes responsive to a patient being late to their appointment. So, for example, when a patient is late, a late pop-up box 705 may show up in the medical/mental health provider's computing device 205. This would then lead to an auto-fill or manual fill pop-up box 510 showing up, and the modified scheduled confirmation 515. The late appointment may be known by the scheduling application by either the medical provider failing to input that the patient has arrived, or by the medical provider inputting into the system that the patient is late or has not arrived. From here, the schedule may either auto-fill or manual fill similarly as discussed with respect to FIGS. 5 and 6. Thus, the system is dynamic in that as soon as a patient is deemed “late” to their appointment, the scheduling application can then immediately, dynamically, and in real-time schedule a new patient's appointment so that patients' get the care they need and a provider's schedule is fully utilized.

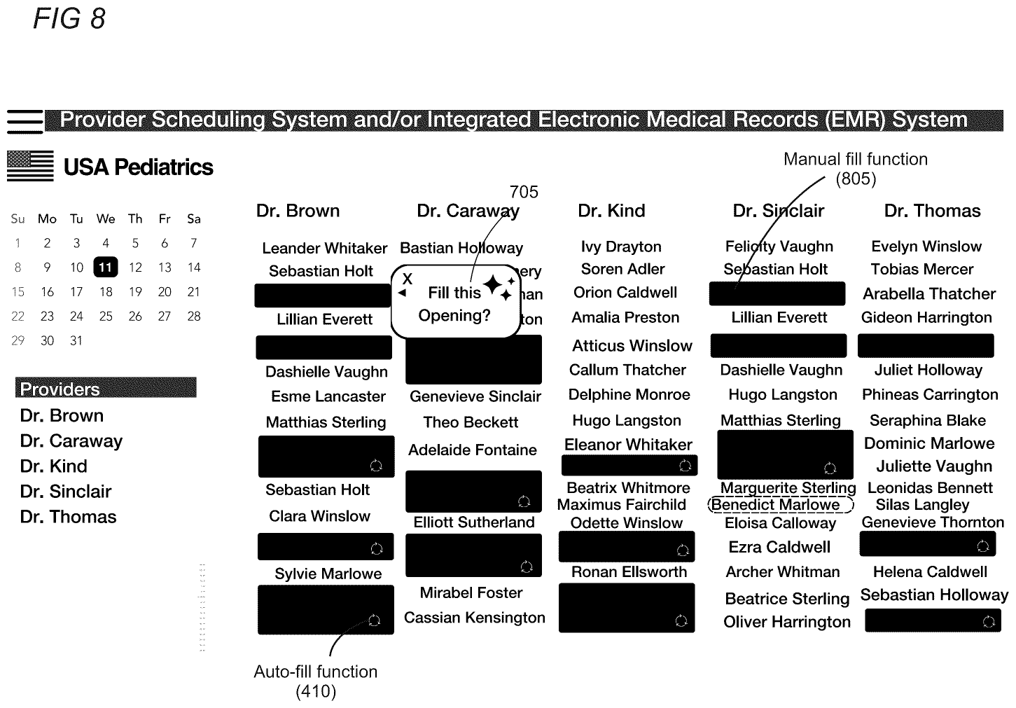

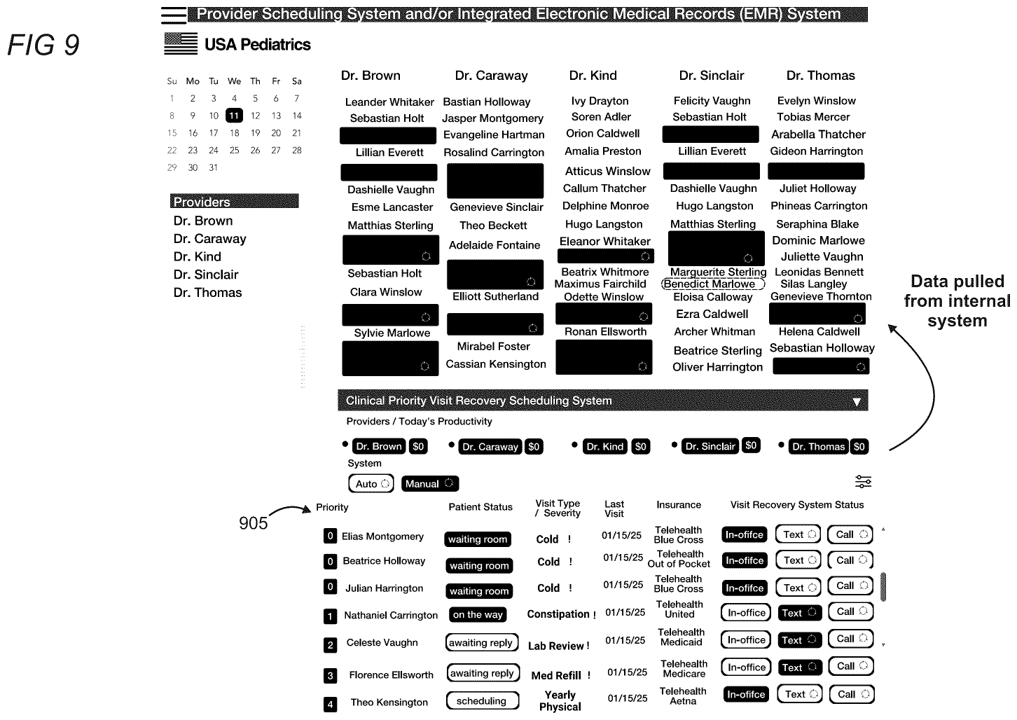

FIG. 8 shows an illustrative representation in which the scheduling application 215 for the medical/mental health provider's computing device 205 can modify the openings 405 to be enabled with either auto-fill function 410 or a manual fill function 805. In this exemplary GUI, the auto-fill function is identifiable with an automated insignia whereas the manual fill option is empty. In other implementations, other insignia or indications can be used to identify auto-fill and manual fill openings.

FIG. 9 shows an illustrative GUI representation in which the scheduling application 215 pulls data from the medical/mental health provider's internal system. Thus, the bottom portion shows a GUI about various patient statuses, including their severity level, so that the scheduling application can make an appropriate determination. Thus, the scheduling application, such as at the internal system # or remote service 220, takes the patient data, their status, severity level, last visit, and other information to propagate a provider's openings 405.

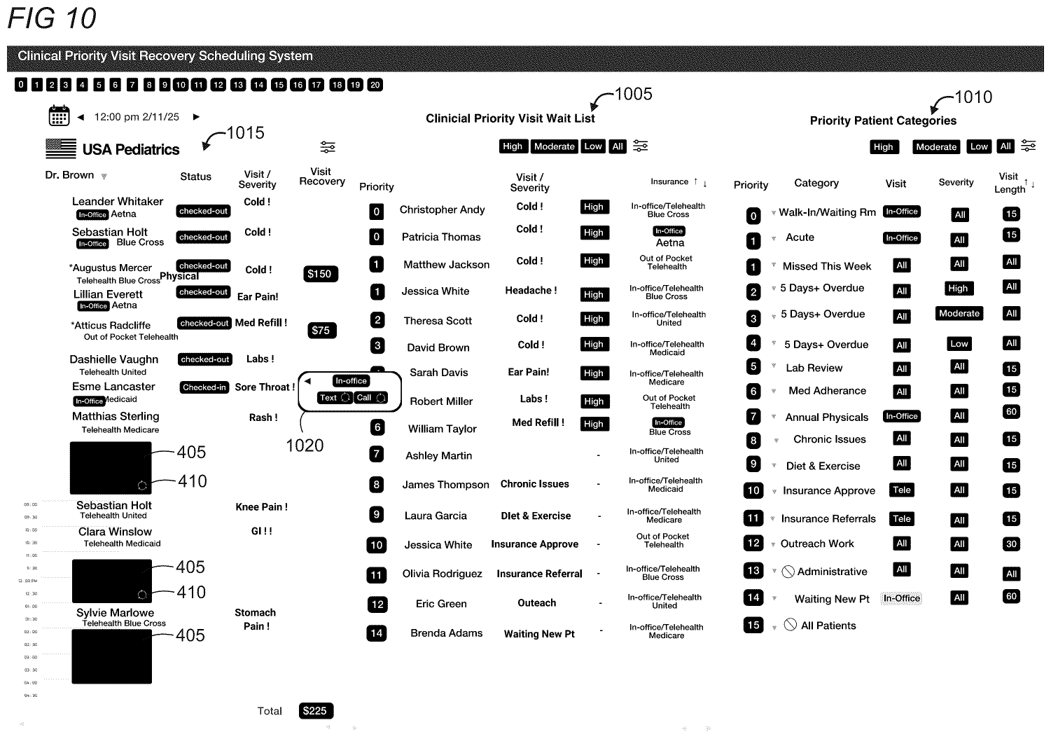

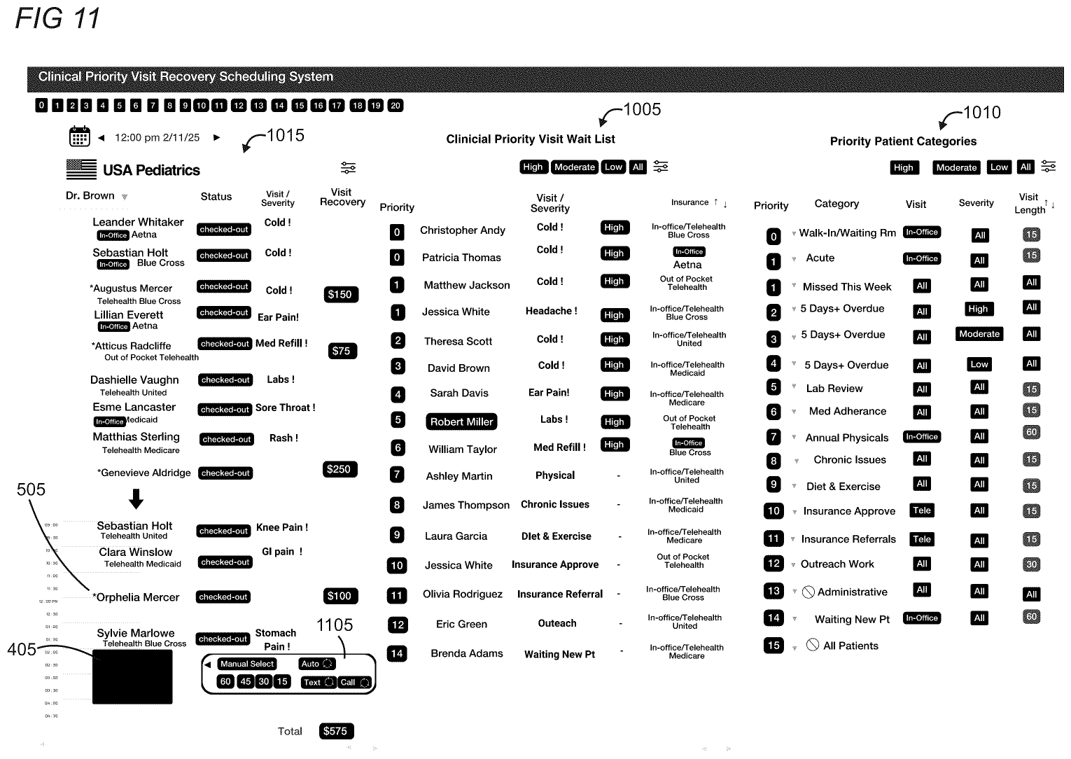

FIG. 10 shows an illustrative GUI representation of a dashboard for a medical/mental health provider computing device 205. Thus, the right column 1010 provides priority patient categories information, which can be broken down by a variety of type of premade, or medical provider created categories, or AI generated categories. For example, as shown, there are categories directed to patients that have “missed sessions this week”, “5 days+overdue patients”, “diet & exercise” patients, patients for “lab review,” etc. Information for each category includes severity level, visit length, type of visit, and a clinical significance for the category of patients, etc. Of note, not shown are categories which can also refer to severity such as “high risk patients” which the medical/mental health provider may wish to keep together to manage and all categories have an option to include or turn off prioritized scheduling systemization. Patient-specific information (e.g., by patient name) can be presented to the medical provider when the click on the down pointing arrow, so they can get a breakdown of patients within that category. A medical/mental health provider is able to move the priority order (e.g., by dragging and dropping). The medical provider is able to find all patient priories based on “high,” “moderate,” “low,” or “all.” Such information conveys to a medical/mental health provider various patient information, severity level, patient tasks to perform, and visitation information. [40] The middle column 1005 provides clinical priority visit wait list information. This column may be prioritized based on the priority patient categories in the right column 1010. Thus, the middle column is specific patients organized for appointments and filling. The middle column includes the specific visit and severity, a priority level based on the severity level (high, medium, low), insurance information, and a patient's last visit (not shown), among other items. In this regard, the insurance information can be utilized because different insurances require different types of visits. So, having an understanding of the patient's severity level, problem, and insurance-type (if any) (and patient's last visit if desired) can help determine an appropriate auto-fill or manual fill propagation into a medical provider's schedule.

The left column 1015 includes, on a high-level, an individual provider's schedule that is being propagated with the middle column 1005 and/or the right column 1010. Thus, the provider's schedule may have openings 405, some of which are configured with the auto-fill functioned 410. Openings without the auto-fill function are manual filled-that is, the provider clicks on and selects the patient, if any, for that time slot (FIG. 6).

FIG. 12 shows an illustrative GUI representation in which the left column 1015 with the provider's schedule has been auto-filled 505 responsive to the auto-fill function being enabled. Thus, the provider's schedule was automatically filled automatically, and the provider can go about their day and see additional patients with little to no work, i.e., zero-touch automation. The remaining opening 405 is still present until the provider clicks the black box (or other opening insignia) and selects a patient (FIG. 6).

FIG. 12 shows an illustrative diagram of a computer system that may be utilized by the computing device, internal system or remote server, periphery device, etc. The architecture 1200 illustrated in FIG. 12 includes one or more processors 1202 (e.g., central processing unit, dedicated Artificial Intelligence chip, graphics processing unit, etc.), a system memory 1204, including RAM (random access memory) 1206, and ROM (read-only memory) 1208, and a system bus 1210 that operatively and functionally couples the components in the architecture 1200. A basic input/output system containing the basic routines that help to transfer information between elements within the architecture 1200, such as during startup, is typically stored in the ROM 1208. The architecture 1200 further includes a mass storage device 1212 for storing software code or other computer-executed code that is utilized to implement applications, the file system, and the operating system. The mass storage device 1212 is connected to the processor 1202 through a mass storage controller (not shown) connected to the bus 1210. The mass storage device 1212 and its associated computer-readable storage media provide non-volatile storage for the architecture 1200. Although the description of computer-readable storage media contained herein refers to a mass storage device, such as a hard disk or CD-ROM drive, it may be appreciated by those skilled in the art that computer-readable storage media can be any available storage media that can be accessed by the architecture 1200.

By way of example, and not limitation, computer-readable storage media may include volatile and non-volatile, removable and non-removable media implemented in any method or technology for storage of information such as computer-readable instructions, data structures, program modules, or other data. For example, computer-readable media includes, but is not limited to, RAM, ROM, EPROM (erasable programmable read-only memory), EEPROM (electrically erasable programmable read-only memory), Flash memory or other solid-state memory technology, CD-ROM, DVD, HD-DVD (High Definition DVD), Blu-ray, or other optical storage, a magnetic cassette, magnetic tape, magnetic disk storage or other magnetic storage device, or any other medium which can be used to store the desired information and which can be accessed by the architecture 1200.

According to various embodiments, the architecture 1200 may operate in a networked environment using logical connections to internal system or remote computers through a network. The architecture 1200 may connect to the network through a network interface unit 1216 connected to the bus 1210. It may be appreciated that the network interface unit 1216 also may be utilized to connect to other types of networks and internal system or remote computer systems. The architecture 1200 also may include an input/output controller 1218 for receiving and processing input from a number of other devices, including a keyboard, mouse, touchpad, touchscreen, control devices such as buttons and switches, or electronic stylus (not shown in FIG. 12). Similarly, the input/output controller 1218 may provide output to a display screen, user interface, a printer, or other output device types (also not shown in FIG. 12).

It may be appreciated that the software components described herein may, when loaded into the processor 1202 and executed, transform the processor 1202 and the overall architecture 1200 from a general-purpose computing system into a special purpose computing system customized to facilitate the functionality presented herein. The processor 1202 may be constructed from any number of transistors or other discrete circuit elements, which may individually or collectively assume any number of states. More specifically, the processor 1202 may operate as a finite-state machine in response to executable instructions contained within the software modules disclosed herein. These computer-executable instructions may transform the processor 1202 by specifying how the processor 1202 transitions between states, thereby transforming the transistors or other discrete hardware elements constituting the processor 1202.

Encoding the software modules presented herein also may transform the physical structure of the computer-readable storage media presented herein. The specific transformation of physical structure may depend on various factors in different implementations of this description. Examples of such factors may include but are not limited to, the technology used to implement the computer-readable storage media, whether the computer-readable storage media is characterized as primary or secondary storage, and the like. For example, if the computer-readable storage media is implemented as semiconductor-based memory, the software disclosed herein may be encoded on the computer-readable storage media by transforming the physical state of the semiconductor memory. For example, the software may transform the state of transistors, capacitors, or other discrete circuit elements constituting the semiconductor memory. The software also may transform the physical state of such components in order to store data thereupon.

As another example, the computer-readable storage media disclosed herein may be implemented using magnetic or optical technology. In such implementations, the software presented herein may transform the physical state of magnetic or optical media when the software is encoded therein. These transformations may include altering the magnetic characteristics of particular locations within given magnetic media. These transformations also may include altering the physical features or characteristics of particular locations within given optical media to change the optical characteristics of those locations. Other transformations of physical media are possible without departing from the scope and spirit of the present description, with the foregoing examples provided only to facilitate this discussion.

The architecture 1200 may further include one or more sensors 1214 or a battery or power supply 1220. The sensors may be coupled to the architecture to pick up data about an environment or a component, including temperature, pressure, etc. Exemplary sensors can include a thermometer, accelerometer, smoke or gas sensor, pressure sensor (barometric or physical), light sensor, ultrasonic sensor, gyroscope, among others. The power supply may be adapted with an AC power cord or a battery, such as a rechargeable battery for portability.

In light of the above, it may be appreciated that many types of physical transformations take place in the architecture 1200 in order to store and execute the software components presented herein. It also may be appreciated that the architecture 1200 may include other types of computing devices, including wearable devices, handheld computers, embedded computer systems, smartphones, PDAs, and other types of computing devices known to those skilled in the art. It is also contemplated that the architecture 1200 may not include all of the components shown in FIG, may include other components that are not explicitly shown in FIG, or may utilize an architecture completely different from that shown in FIG.

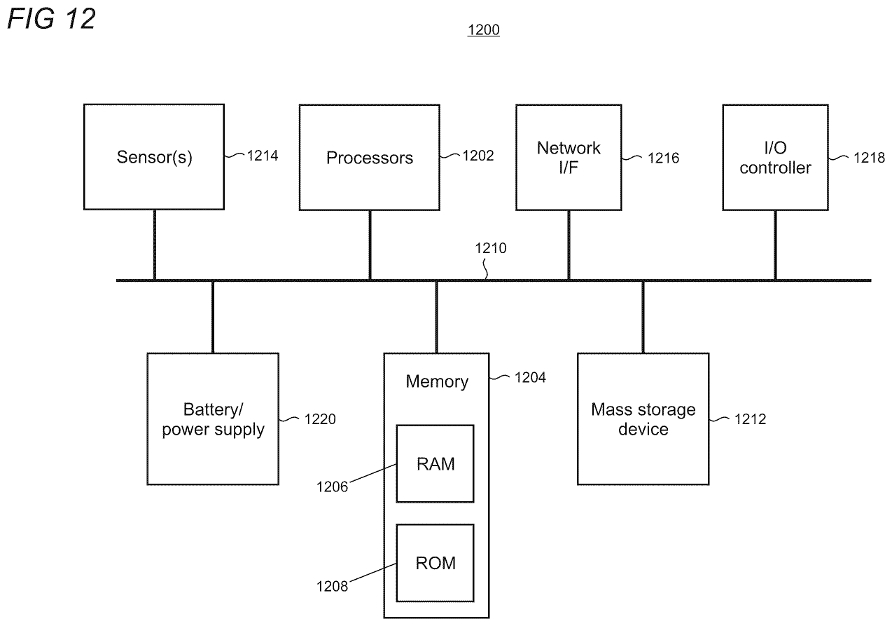

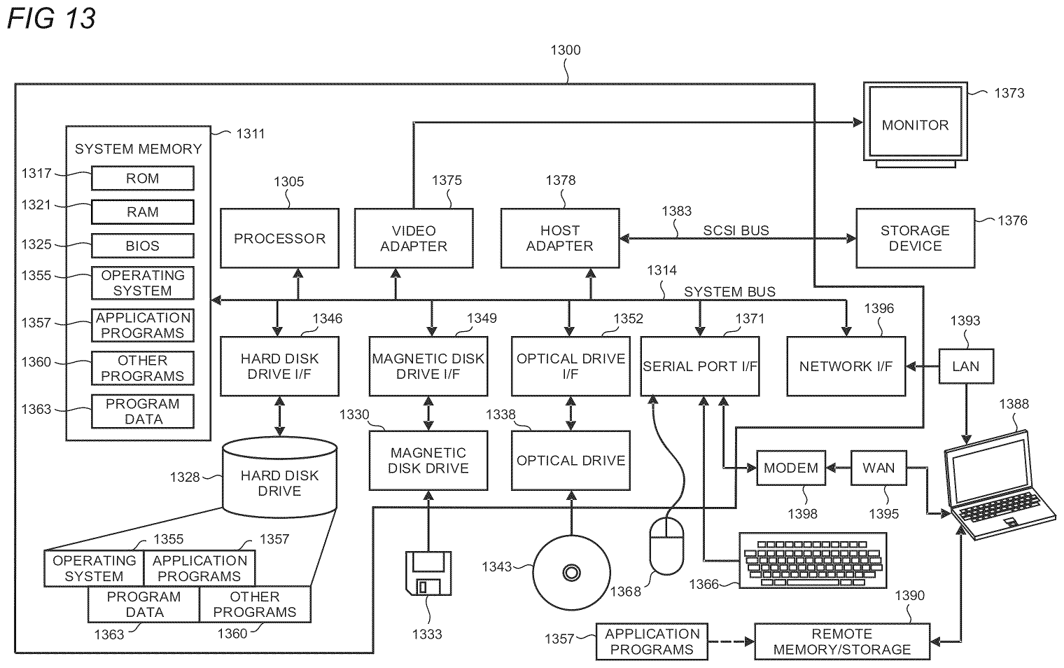

FIG. 13 is a simplified block diagram of an illustrative computer system 1300 such as a smartphone, PC (personal computer), laptop computer, or server with which the present scheduling application may be implemented.

Computer system 1300 includes a processor 1305, a system memory 1311, and a system bus 1314 that couples various system components including the system memory 1311 to the processor 1305. The system bus 1314 may be any of several types of bus structures, including a memory bus or memory controller, a peripheral bus, or a local bus using any of a variety of bus architectures. The system memory 1311 includes read-only memory (ROM) 1317 and random-access memory (RAM) 1321. A basic input/output system (BIOS) 1325, containing the basic routines that help to transfer information between elements within the computer system 1300, such as during startup, is stored in ROM 1317. The computer system 1300 may further include a hard disk drive 1328 for reading from and writing to an internally disposed hard disk (not shown), a magnetic disk drive 1330 for reading from, or writing to a removable magnetic disk 1333 (e.g., a floppy disk), and an optical disk drive 1338 for reading from or writing to a removable optical disk 1343 such as a CD (compact disc), DVD (digital versatile disc), or other optical media. The hard disk drive 1328, magnetic disk drive 1330, and optical disk drive 1338 are connected to the system bus 1314 by a hard disk drive interface 1346, a magnetic disk drive interface 1349, and an optical drive interface 1352, respectively. The drives and their associated computer-readable storage media provide non-volatile storage of computer-readable instructions, data structures, program modules, and other data for the computer system 1300. Although this illustrative example includes a hard disk, a removable magnetic disk 1333, and a removable optical disk 1343, other types of computer-readable storage media which can store data that is accessible by a computer such as magnetic cassettes, Flash memory cards, digital video disks, data cartridges, random access memories (RAMs), read-only memories (ROMs), and the like may also be used in some applications of the present scheduling application. In addition, as used herein, the term computer-readable storage media includes one or more instances of a media type (e.g., one or more magnetic disks, one or more CDs, etc.). For purposes of this specification and the claims, the phrase “computer-readable storage media” and variations thereof are intended to cover non-transitory embodiments and do not include waves, signals, and/or other transitory and/or intangible communication media.

A number of program modules may be stored on the hard disk, magnetic disk 1333, optical disk 1343, ROM 1317, or RAM 1321, including an operating system 1355, one or more application programs 1357, other program modules 1360, and program data 1363. A user may enter commands and information into the computer system 1300 through input devices such as a keyboard 1366 and pointing device 1368 such as a mouse. Other input devices (not shown) may include a microphone, joystick, gamepad, satellite dish, scanner, trackball, touchpad, touchscreen, touch-sensitive device, voice-command module or device, user motion or user gesture capture device, or the like. These and other input devices are often connected to the processor 1305 through a serial port interface 1371 that is coupled to the system bus 1314 but may be connected by other interfaces, such as a parallel port, game port, or universal serial bus (USB). A monitor 1373 or other type of display device is also connected to the system bus 1314 via an interface, such as a video adapter 1375. In addition to the monitor 1373, personal computers typically include other peripheral output devices (not shown), such as speakers and printers. The illustrative example shown in FIG. 13 also includes a host adapter 1378, a Small Computer System Interface (SCSI) bus 1383, and an external storage device 1376 connected to the SCSI bus 1383.

The computer system 1300 is operable in a networked environment using logical connections to one or more remote computers, such as a remote computer 1388. The remote computer 1388 may be selected as another personal computer, a server, a router, a network PC, a peer device, or other common network nodes, and typically includes many or all of the elements described above relative to the computer system 1300, although only a single representative remote memory/storage device 1390 is shown in FIG. 13. The logical connections depicted in FIG. 13 include a local area network (LAN) 1393 and a wide area network (WAN) 1395. Such networking environments are often deployed, for example, in offices, enterprise-wide computer networks, intranets, and the Internet.

When used in a LAN networking environment, the computer system 1300 is connected to the local area network 1393 through a network interface or adapter 1396. When used in a WAN networking environment, the computer system 1300 typically includes a broadband modem 1398, network gateway, or other means for establishing communications over the wide area network 1395, such as the Internet. The broadband modem 1398, which may be internal or external, is connected to the system bus 1314 via a serial port interface 1371. In a networked environment, program modules related to the computer system 1300, or portions thereof, may be stored in the remote memory storage device 1390. It is noted that the network connections shown in FIG. 13 are illustrative, and other means of establishing a communications link between the computers may be used depending on the specific requirements of an application of the present scheduling application.

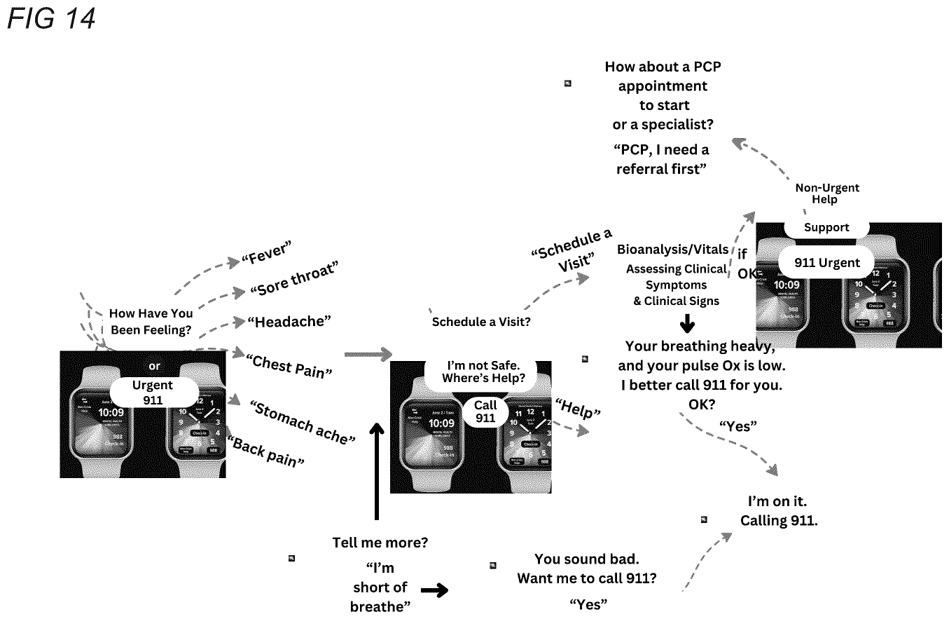

FIGS. 14-17 show illustrative representations in which a patient operating a computing device, such as a smartwatch, retrieving clinical factor information for a given patient and scheduling visits or at least communicating with medical providers. While a smartwatch is shown in FIGS. 14-17, that is exemplary only and other computing devices are also possible, such as laptop computers, personal computers, tablet computers, head-mounted display (HMD) devices, etc.

Referring first to FIG. 14, the patient-user may be prompted by their smartwatch about symptoms. User input about their symptoms, whether input physically on a keyboard, touchscreen, voice-interaction, and the like, may prompt multiple back-and-forth interactions with the smartwatch. Such communications and/or user input may help provide severity information to the remote service's or internal system's scheduling application 215.

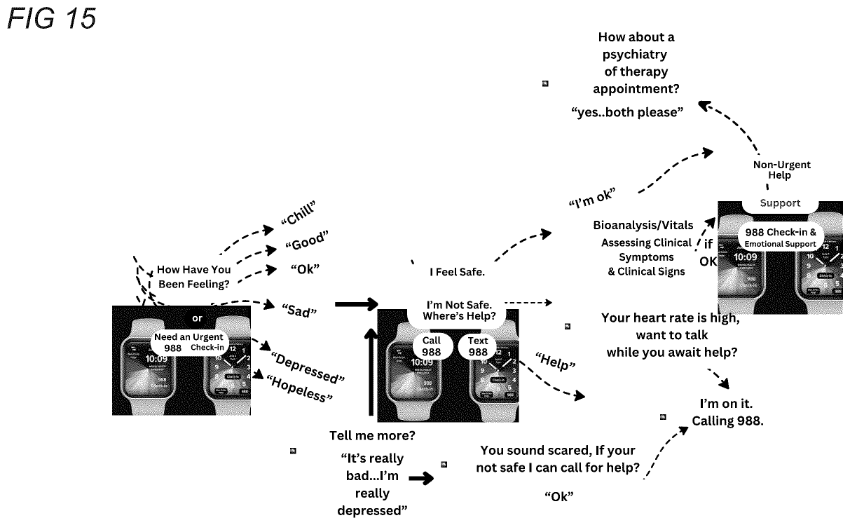

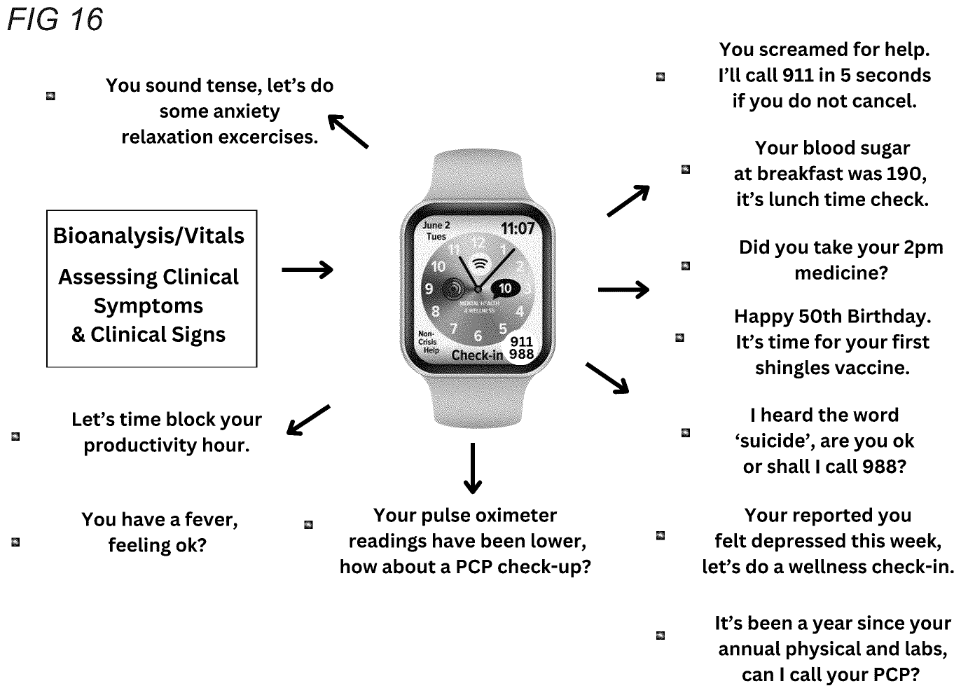

FIGS. 15-16 shows an illustrative representation in which the smartwatch, utilizing internal sensors or other information about the user, questions or alerts the user about various symptoms or concerns. This may prompt the user-patient to respond to the smartwatch and either input information, provide additional feedback, answer questions, or seek help. Such input, rated from low to high severity, can be transmitted to the scheduling application 215 at the remote service 220 or internal medical provider's service 275 for utilization. Such data can be used in the scheduling application.

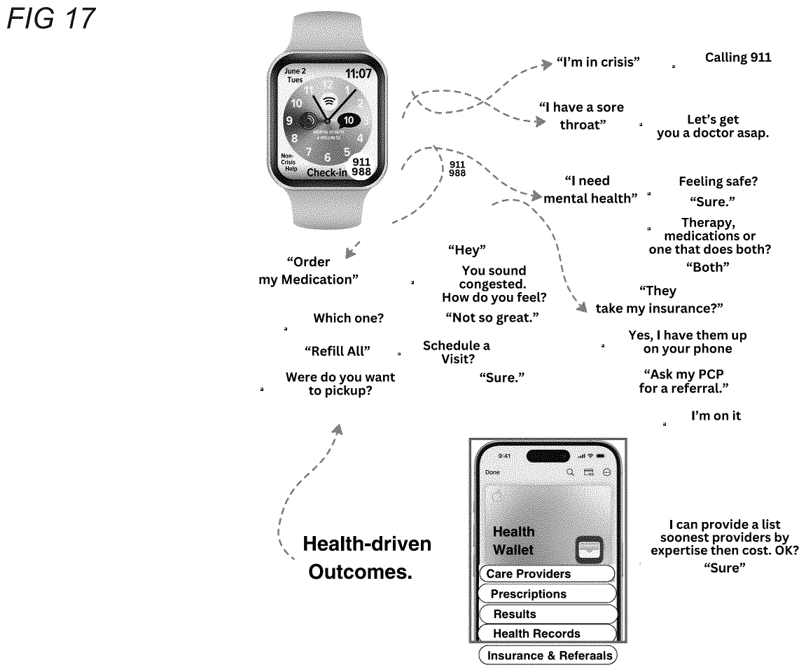

Referring to FIG. 16, the smartwatch triggers various information and questions to the user, whether specific to the user's physiological data or other known information about the user, like their medication time, birthday or other personal attributes, etc. Such information can be utilized by the scheduling application 215 when scheduling a provider visit. FIG. 17 provides additional outputs and inputs between the patient-user and the smartwatch, which may be utilized by the scheduling application 215, such as on the dashboard, automated or manual scheduling functions, etc.

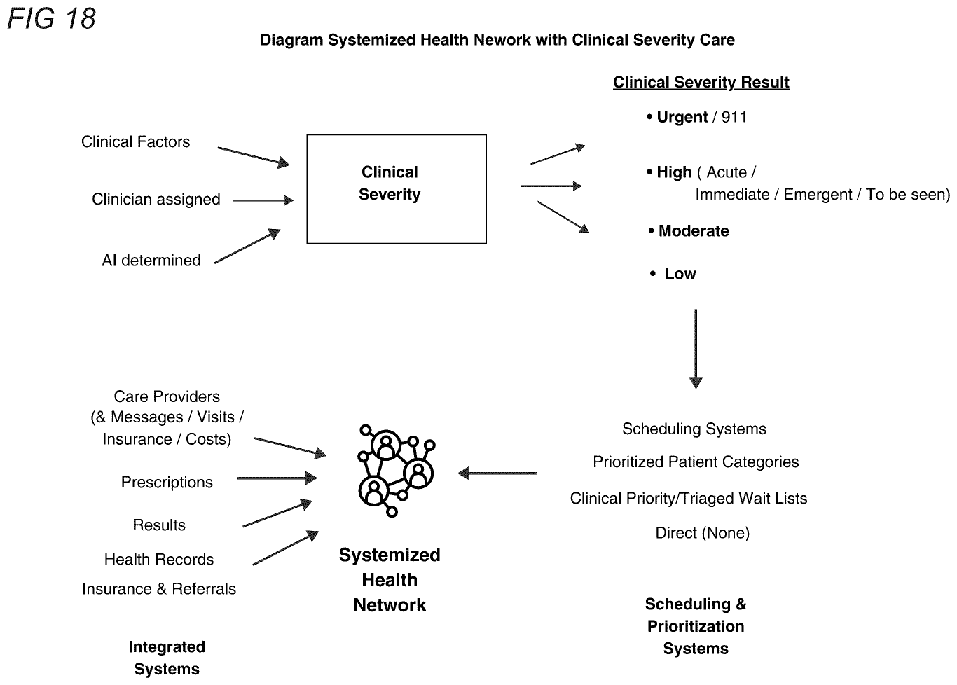

FIG. 18 shows an illustrative environment of the overall systemized health network with clinical severity care. The diagram shows a high-level overview of the disclosure herein, from the clinical factors, to the clinical severity, result, scheduling system and dashboard information, systemized healthcare network, and ultimately the integrated systems.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computing device configured with a scheduling application, comprising: one or more processors; and
one or more hardware-based memory devices having computer-executable instructions which, when executed by the one or more processors, causes the computing device to:
provide a GUI (graphical user interface) of a schedule, the schedule including
rows and columns for one or more providers and their appointments for a given day; indicate, on the schedule for each of the one or more providers, openings, the openings being unfilled appointment times;
fill an opening on a provider's schedule; and
device indicating their availability overlaps with the opening on the provider's schedule.

2. The computing device of claim 1, wherein the filling of the empty slot occurs automatically.

3. The computing device of claim 1, wherein the computing device is further configured to:
determine a severity for each patient; and automatically place the patient with a greater severity in the empty slot.

4. The computing device of claim 1, wherein the scheduling application receives user input to fill a specific opening in their schedule, and the computing device presents multiple patients for manual selection by the provider or provider's staff.

5. The computing device of claim 4, wherein the presented multiple patients includes a severity level for each patient.

6. The computing device of claim 5, wherein the provider selects a specific patient of the presented multiple patients.

7. The computing device of claim 1, wherein a GUI on the computing device presents indicators that distinguish between automatically and manually filled openings on the schedule.

8. One or more hardware-based non-transitory computing devices including instructions disposed within a computing device which, when executed by the one or more processors, cause the computing device to:
provide a GUI (graphical user interface) of a schedule, the schedule including
rows and columns for one or more providers and their appointments for a given day; indicate, on the schedule for each of the one or more providers, openings, the
openings being unfilled appointment times; and
fill an opening on a provider's schedule;
device indicating their availability overlaps with the opening on the provider's schedule.

9. The one or more hardware-based memory devices of claim 8, wherein the filling of the empty slot occurs automatically.

10. The one or more hardware-based memory devices of claim 8, wherein the computing device is further configured to:
determine a severity for each patient; and automatically place the patient with a greater severity in the empty slot.

11. The one or more hardware-based memory devices of claim 8, wherein the scheduling application receives user input to fill a specific opening in their schedule, and the computing device presents multiple patients for manual selection by the provider.

12. The one or more hardware-based memory devices of claim 11, wherein the presented multiple patients includes a severity level for each patient.

13. The one or more hardware-based memory devices of claim 12, wherein the provider selects a specific patient of the presented multiple patients.

14. The one or more hardware-based memory devices of claim 8, wherein a GUI on the computing device presents indicators that distinguish between automatically and manually filled openings on the schedule.

15. A method performed by a computing device, comprising:
providing a GUI (graphical user interface) of a schedule, the schedule including
rows and columns for one or more providers and their appointments for a given day; indicating, on the schedule for each of the one or more providers, openings, the
openings being unfilled appointment times; and
filling an opening on a provider's schedule; and
device indicating their availability overlaps with the opening on the provider's schedule.

16. The method of claim 15, wherein the filling of the empty slot occurs automatically.

17. The method device of claim 15, wherein the computing device is further configured to:
determine a severity for each patient; and automatically place the patient with a greater severity in the empty slot.

18. The method of claim 15, wherein the scheduling application receives user input to fill a specific opening in their schedule, and the computing device presents multiple patients for manual selection by the provider.

19. The method of claim 18, wherein the presented multiple patients includes a severity level for each patient.

20. The method of claim 19, wherein the provider selects a specific patient of the presented multiple patients.
